(19) United States
(12) Patent Application Publication
(10) Pub. No.: US 2026/0047788 A1
(43) Pub. Date: Feb. 19, 2026

(54) SYSTEM

(71) Applicant: SOFTBANK GROUP CORP., Tokyo (JP)

(72) Inventor: Fumika BEPPU, Tokyo (JP)

(73) Assignee: SOFTBANK GROUP CORP., Tokyo (JP)

(21) Appl. No.: 19/299,495

(22) Filed: Aug. 14, 2025

(30) Foreign Application Priority Data
Aug. 16, 2024 (JP) 2024-137238

(51) Int. Cl. A61B 5/16; A61B 5/00; A61B 5/372

(52) U.S. Cl. CPC A61B 5/165; A61B 5/0006; A61B 5/372; A61B 5/7203; A61B 5/7267

(57) ABSTRACT

System includes a processor that is configured to: collect EEG data obtained by an EEG measurement device, preprocess the collected EEG data and convert the data into a format suitable for analysis, input the preprocessed EEG data into a deep learning model to analyze emotions or thoughts, convert the analysis result into natural language using a generative AI model, and display the converted natural language.

### CROSS-REFERENCE TO RELATED APPLICATION

This application claims priority under 35 USC 119 from Japanese Patent Application No. 2024-137238 filed Aug. 16, 2024, the disclosure of which is incorporated by reference herein in its entirety.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

In medical and caregiving environments, it is often extremely challenging for caregivers or healthcare providers to accurately understand the emotions and thoughts of patients or care recipients who have difficulties in verbal communication, such as those with neurological disorders or severe disabilities. Conventional communication aids are limited in their ability to interpret non-verbal cues, which can hinder the provision of appropriate care and timely response. There is a need for a system that can interpret a user's internal emotional or cognitive state in real time and effectively relay this information to caregivers using natural language output.

### SUMMARY

In order to address the above challenges, the present invention provides a system including a processor, wherein the processor collects EEG data obtained by an EEG measurement device, preprocesses the collected EEG data and converts it into a format suitable for analysis, inputs the preprocessed EEG data into a deep learning model to analyze emotions or thoughts, converts the analysis result into natural language using a generative AI model, and displays the converted natural language. The processor may further transmit the collected EEG data via a secure communication protocol and apply a noise removal filter during the preprocessing stage. By implementing these steps, the invention enables real-time, accurate, and secure analysis and communication of a user's emotional or cognitive state, thereby improving interaction and care quality in medical and caregiving settings.

“EEG data” means electrical signals generated by brain activity and measured by an electroencephalography (EEG) device, typically representing voltage fluctuations recorded at the scalp.

“EEG measurement device” means a medical device designed to non-invasively detect and record electrical activity of the brain from the surface of the scalp.

“Processor” means a computational unit, such as a microprocessor or microcontroller, that executes software instructions to perform data processing and control functions within the system.

“Preprocessing” means a series of computational operations applied to raw EEG data to remove noise, artifacts, or irrelevant segments and to convert the data into a usable format for subsequent analysis.

“Deep learning model” means an artificial intelligence model utilizing layered neural networks, such as convolutional or recurrent neural networks, which is trained to recognize patterns and classify data, for example, to infer emotions or thoughts from EEG signals.

“Generative AI model” means an artificial intelligence model designed to generate human-like natural language text based on given inputs, such as a detected emotional or cognitive state.

“Secure communication protocol” means a standardized method or set of rules (e.g., HTTPS) used for encrypting and transmitting data safely over a network to prevent unauthorized access or modification.

“Noise removal filter” means a signal processing algorithm or method used to reduce or eliminate unwanted noise and artifacts from EEG data, thereby enhancing its quality and reliability for further analysis.

“Natural language” means human language that is used for everyday communication, including written or spoken expressions, as opposed to machine or code language.

“Display” means presenting information, such as natural language text, visually on a monitor, screen, or other output device, so that it can be recognized and understood by users, including caregivers or healthcare providers.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions; and

FIG. 10 illustrates an emotion map mapping plural emotions.

FIG. 11 is a sequence diagram showing the flow of data processing system processing in Example 1.

FIG. 12 is a sequence diagram showing the flow of data processing system processing in Application Example 1.

FIG. 13 is a sequence diagram showing the flow of data processing system processing in Example 2.

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings. First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

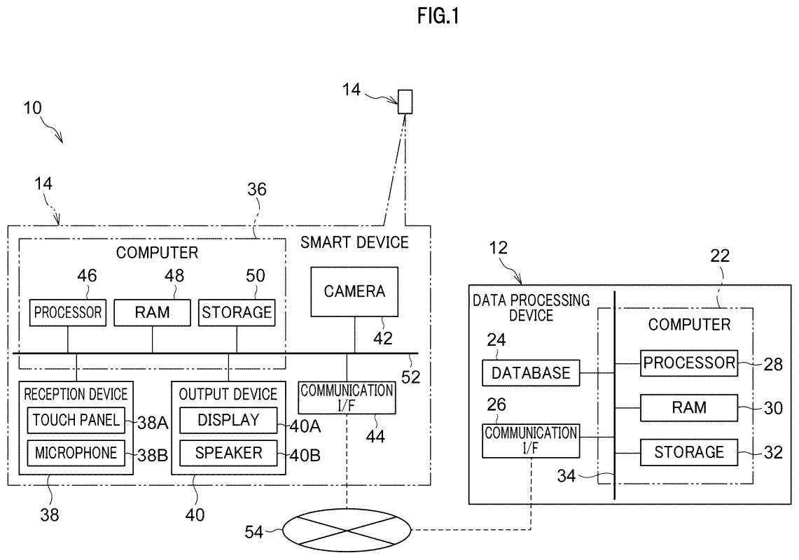

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

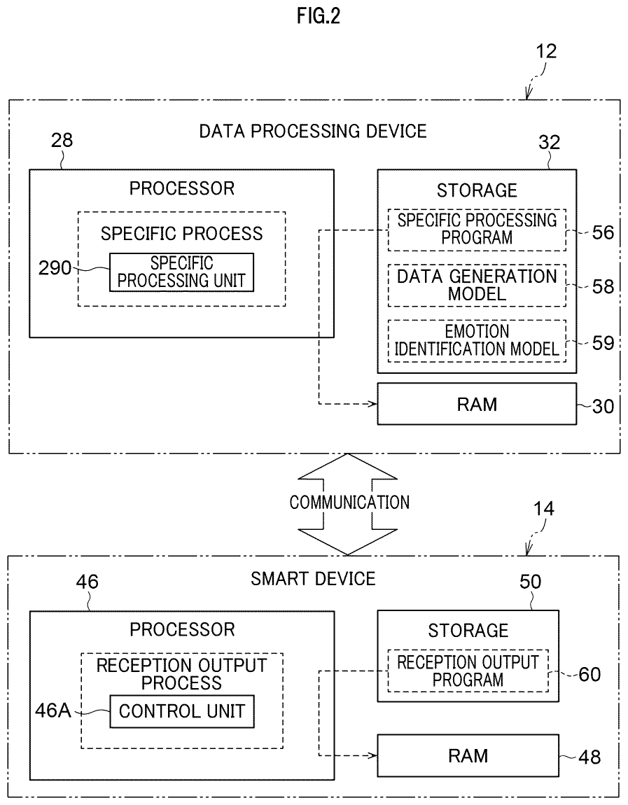

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional biological signal analysis systems, particularly those for brainwave data, typically require multiple manual interventions for data acquisition, preprocessing, analysis, and result presentation, leading to delays and limited real-time usability in medical or nursing environments. In addition, issues such as poor data security during communication, inadequate noise removal, low automation in data-to-language conversion, and lack of reliability in the output have hindered practical application, especially for supporting communication with patients or care-dependent individuals who cannot easily express their states.

The specific processing by the specific processing unit 290 of the data processing device 12 in Example 1 is realized by the following means.

The present invention provides a server including a processor configured to collect biological signal data obtained by a biosignal acquisition device, temporarily store and manage the data as time-series data, transmit the encrypted data to an information processing apparatus using a secure communication protocol, verify and decrypt the received data, preprocess the data including noise removal, analyze the preprocessed data with a machine learning model to classify the mental or cognitive state, generate a prompt sentence based on the classification, input the prompt and classification into a generative artificial intelligence model to produce a natural language message, and transmit and display the generated message on a display device. This enables secure, high-precision, and rapid analysis of biological signals such as brainwaves and reliable conversion of the analytical results into natural language, thereby enhancing communication and response efficiency in medical and nursing care settings.

The term “biosignal acquisition device” refers to an apparatus configured to detect, measure, and output physiological signals, such as brainwaves or other electrical activities generated by a living body.

The term “biological signal data” refers to digitized information representing physiological activities collected from a user via a biosignal acquisition device.

The term “storage device” refers to an electronic component or subsystem used to temporarily or permanently retain digital data for subsequent processing.

The term “time-series data” refers to a sequence of data points representing measurements taken at consecutive, uniformly spaced points in time.

The term “secure communication protocol” refers to a digital communication method that ensures data confidentiality, integrity, and authenticity during transmission, such as encrypted protocols.

The term “information processing apparatus” refers to a hardware and/or software system configured to process, analyze, and manage digital data, including servers or computing devices.

The term “integrity verification” refers to the process of checking whether transmitted or received data has been altered or corrupted during transfer.

The term “decryption” refers to the process of converting encrypted data back into its original, intelligible form using a cryptographic method.

The term “preprocessing” refers to a series of data manipulation steps, including noise removal and data formatting, to prepare raw data for further analysis.

The term “noise removal” refers to the application of digital filters or algorithms designed to eliminate undesired artifacts or interference from raw biological signal data.

The term “analyzable data format” refers to a digital representation of data that is structured and conditioned for compatibility with computational analysis methods.

The term “machine learning model” refers to a computational model trained on data to recognize patterns, classify inputs, or make predictions regarding new data.

The term “mental state or cognitive state” refers to the assessment results indicating the psychological or intellectual condition of a user as inferred from biological signals.

The term “prompt sentence” refers to a structured input statement or instruction provided to a generative artificial intelligence model to guide the generation of natural language output.

The term “generative artificial intelligence model” refers to a computational system capable of producing or transforming textual content, such as natural language sentences, based on prompt sentences and input data.

The term “natural language message” refers to a text output generated in a form understandable by humans and expressing information derived from system analysis.

The term “display device” refers to an electronic apparatus, such as a monitor or screen, used for presenting text or graphical information to a user.

The system includes a server, one or more terminals, and a biosignal acquisition device such as an EEG headset. The biosignal acquisition device may be any commercially available device capable of capturing electrical activity from the human body, for example, an EEG device with multiple electrodes. The terminal can be a smartphone, a tablet, or a dedicated computing device equipped with wireless communication functionality (such as Bluetooth or Wi-Fi) and a display.

The terminal is configured to establish a wireless connection with the biosignal acquisition device and to continuously collect biological signal data from the user. The terminal temporarily stores the collected data in a storage device (such as RAM or local storage), managing it as time-series data. The terminal then encrypts the biological signal data utilizing standard cryptographic techniques, for example, the Advanced Encryption Standard (AES). The terminal uses a secure communication protocol, such as HTTPS or SSL/TLS, to transmit the encrypted data to the server. During this process, the terminal also monitors the integrity of communication and handles retransmission in case of communication failure.

The server, which is implemented as an information processing apparatus such as a cloud-based or on-premises computing unit, receives the encrypted biological signal data through its network interface. The server verifies the integrity of the received data and decrypts it with a preapproved cryptographic key. The decrypted data is then stored temporarily as an analyzable array, for example, in a NumPy array if the server is running Python.

To remove noise and preprocessing artifacts, the server applies a digital filter such as a Butterworth filter using a software library like SciPy. The server then converts the cleaned data into a format ready for computational analysis. For machine learning-based classification of the user's mental or cognitive state, the server uses a pre-trained machine learning model. Suitable platforms for model execution include TensorFlow or PyTorch, and the model may be trained beforehand on large datasets of biosignal data labeled with specific emotional or cognitive states.

Once the server classifies the current mental or cognitive state of the user (such as “anxiety,” “joy,” or “sadness”), the server generates a prompt sentence tailored to the result. The prompt sentence is constructed in natural language so that a generative AI model can convert the classified state into a personalized, human-like message. For example, if the classified state is “anxiety,” the prompt sentence may be:

“The user's mental state has been classified as ‘anxiety.’ Please generate a message explaining this sentiment in a way that the user might express.”

Alternatively, in another language:

“Hikaigosha no kanjou ga “fuan” to kaiseki saremashita. Kono kanjou wo setsumei suru bunshou wo seisei shite kudasai.”

The server then sends the prompt sentence, along with the classification, to a generative artificial intelligence model, such as a large language model accessible via an API. The generative AI model processes the input and generates a natural language message corresponding to the user's mental or cognitive state, such as:

“I feel anxious. There is something I am worried about.”

The server then encrypts this generated natural language message and returns it to the terminal using the secure communication protocol. The terminal receives the message, decrypts it, and displays it on the integrated display for the user, caregiver, or medical professional to read. The user, such as a caregiver, may then use the information presented in the message to communicate with or provide appropriate care to the individual from whom the biological signal was obtained.

The software modules involved in the system may include the SDK provided by the biosignal device manufacturer for data acquisition, encryption/decryption libraries for security, network protocols for secure communication, Python libraries such as NumPy and SciPy for data manipulation and filtering, TensorFlow or PyTorch for running the machine learning model, and an interface to the generative AI model, for instance, via a standard REST API.

Through the above configuration, the invention enables real-time, secure, and automated analysis of biological signals and the delivery of human-comprehensible messages generated by a generative artificial intelligence model, based on prompt sentences automatically constructed for the given user state. This allows for immediate and accurate communication support in medical or caregiving environments.

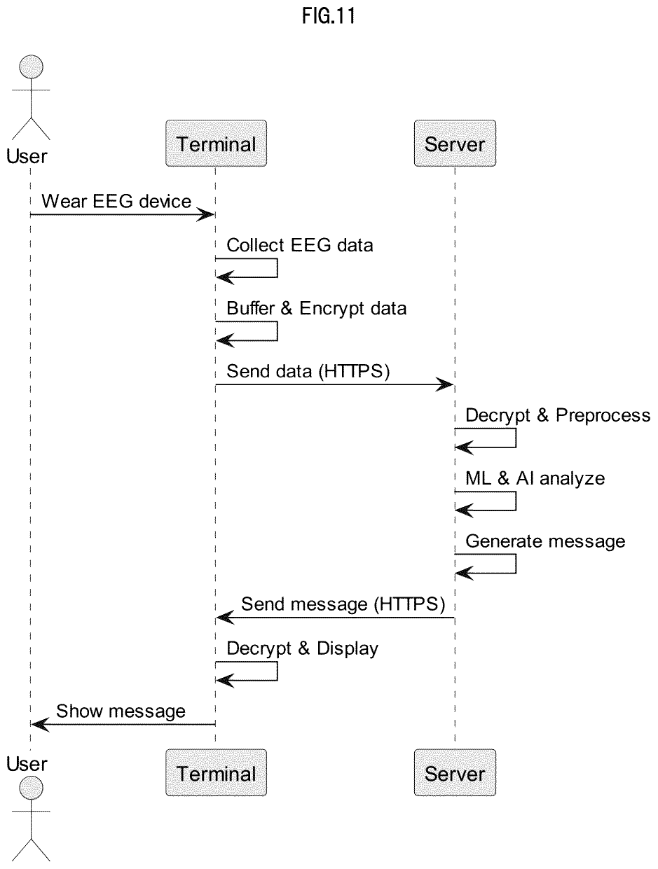

The following describes the processing flow using FIG. 11.

### Step 1:

The terminal establishes a wireless connection with the biosignal acquisition device using Bluetooth or Wi-Fi. The terminal receives biological signal data, such as EEG signals, from the biosignal acquisition device at a defined sampling rate (for example, 256 Hz). The input for this step is real-time analog signals from the user's body, which are converted to digital data by the biosignal acquisition device and transmitted to the terminal. The terminal then stores the collected digital biological signal data in its local memory buffer as time-series data. The output of this step is the buffered biological signal data in digital format.

### Step 2:

The terminal monitors the buffer size and when a predefined amount of data (for instance, 5 seconds' worth) is accumulated, the terminal encrypts the buffered biological signal data using a symmetric encryption algorithm such as AES. As input, the terminal uses the buffered biological signal data. The terminal formats the encrypted data into a structured data format, such as JSON, attaching metadata including timestamps and device IDs. The output is a securely encrypted data packet ready for network transmission.

### Step 3:

The terminal establishes a secure communication channel with the server using a protocol such as HTTPS or SSL/TLS. The terminal transmits the encrypted biological signal data packet to the server and waits for an acknowledgement. The input is the encrypted data packet from Step 2. The terminal manages potential transmission failures by automatically retrying the connection or resending the data. The output from this step is the successful delivery of the encrypted data packet to the server.

### Step 4:

The server receives the encrypted biological signal data packet via its network interface. The server verifies the integrity of the received packet by checking sequence numbers, timestamps, and digital signatures in the packet. The input to this step is the encrypted data packet received from the terminal. The server then decrypts the data using a pre-shared symmetric key. The output is the raw, decrypted biological signal data, organized as a digital time-series array.

### Step 5:

The server preprocesses the obtained biological signal data by applying a digital filter, such as a Butterworth bandpass filter using a signal processing library. The input is the raw time-series biological signal data. The server applies noise removal by filtering out unwanted frequency components and artifacts. The output is a cleaned, denoised biological signal dataset, suitable for subsequent analysis.

### Step 6:

The server normalizes the cleaned signal data and reshapes it according to the requirements of a pre-trained machine learning model, for example, converting it to a multi-dimensional array. The input is the denoised biological signal dataset from Step 5. The server runs inference using a machine learning model, implemented using frameworks such as TensorFlow or PyTorch, to classify the user's mental or cognitive state (such as “anxiety,” “joy,” or “sadness”). The output is a classification result label and a confidence score.

### Step 7:

The server generates a prompt sentence based on the classification result from the previous step. For example, when the classification result is “anxiety,” the server creates a prompt such as: “The user's mental state has been classified as ‘anxiety.’ Please generate a message explaining this sentiment as if the user is speaking.” The input for this step is the classification label. The server passes the prompt sentence and classification result to a generative artificial intelligence model via API (such as a cloud LLM endpoint). The output from this step is a generated natural language message reflecting the user's detected state.

### Step 8:

The server encrypts the generated natural language message using a secure encryption method. The input for this step is the natural language message generated by the AI model. The server then transmits the encrypted message back to the terminal via HTTPS or SSL/TLS. The output is the successful, secure delivery of the message to the terminal.

### Step 9:

The terminal receives the encrypted natural language message from the server and decrypts it using the appropriate key. The input is the encrypted natural language message. The terminal processes and formats the decrypted message appropriately for its graphical user interface. The terminal then displays the natural language message clearly on its screen. The output is the visible display of the natural language message on the terminal's display device for the user to interpret and act upon.

### Step 10:

The user, such as a caregiver or health professional, reads the displayed natural language message on the terminal. The input is the message shown on the screen. Based on the content, such as “I feel anxious. There is something I am worried about,” the user may communicate with the individual or adjust their care accordingly. The output is an informed action or response from the user, facilitated by the system's ability to deliver precise and timely information.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

In conventional environments such as healthcare, caregiving, and in-person customer service, it has been extremely difficult to accurately and efficiently understand a subject's emotional or cognitive state in real time. This limitation has often caused delays or inaccuracies in providing appropriate care, support, or personalized responses, particularly when subjects have communication difficulties. Furthermore, there is a need for a system that can securely process and transmit sensitive biological signals, integrate multimodal data, and effectively convert complex analytical results into human-understandable natural language, while ensuring information privacy and integrity.

The specific processing by the specific processing unit 290 of the data processing device 12 in Application Example 1 is realized by the following means.

The present invention provides a server comprising a processor configured to collect biological signal data from a biological signal acquisition device, preprocess and clean the biological signal data by applying signal processing techniques, analyze a state of a subject by inputting the processed data into a machine learning model, integrate additional sensor data including image, voice, and motion information to improve analysis accuracy, input the analyzed state information and supplementary emotional information as prompt sentences into a generative information generation model to convert the data into natural language, and display the natural language output on an output device, with at least one of the biological signal data and natural language information being transmitted and received using an encryption-based communication method. This enables real-time, accurate, and secure understanding and communication of a subject's emotional or cognitive state in various environments such as healthcare, caregiving, and customer service, thereby supporting timely and appropriate responses and improving the quality of interaction.

The term “biological signal acquisition device” refers to a device that is capable of collecting physiological signals from a subject, such as electrical signals generated by the brain, muscles, heart, or other organs.

The term “biological signal data” refers to digital or analog data representing physiological signals captured from a subject by a biological signal acquisition device.

The term “preprocess” refers to performing initial data manipulation and transformation steps on raw data to prepare it for further analysis, which may include filtering, normalization, segmentation, and conversion to a suitable format.

The term “signal processing techniques” refers to mathematical and computational methods applied to biological signal data in order to remove noise, correct artifacts, and extract relevant features for subsequent analysis.

The term “machine learning model” refers to a computational model, such as a trained algorithm or neural network, that is capable of analyzing input data to recognize patterns, classify states, or make predictions based on learned relationships.

The term “state of a subject” refers to the physical, emotional, or cognitive condition or status of an individual as determined by analyzing biological signal data and other sensor information.

The term “sensor data” refers to information obtained from various sensing devices, including but not limited to image sensors, audio sensors, and motion sensors.

The term “generative information generation model” refers to an artificial intelligence model capable of producing or transforming data into human-understandable output, such as generating natural language descriptions based on input information.

The term “prompt sentence” refers to a structured input or command fed into a generative information generation model, specifying the content or context to be expressed in the generated output.

The term “natural language” refers to language that is used by humans for everyday communication, such as English, Japanese, or other spoken or written languages.

The term “output device” refers to any device capable of displaying or presenting information to a user, including visual displays, monitors, wearable devices, or auditory output devices.

The term “encryption-based communication method” refers to any data transmission protocol or method that employs encryption techniques to ensure confidentiality, integrity, and security of transmitted information.

The system includes a processor, which may be deployed on a server, client terminal, or a combination thereof. The hardware configuration includes at least a biological signal acquisition device (for example, an EEG headset or sensor-equipped wearable device), a communication interface supporting encryption-based protocols (such as HTTPS or MQTT), output devices (such as smart glasses displays, tablets, or dedicated monitors), and the computing infrastructure necessary to execute the software components.

Software modules used in the system include a signal processing library (such as NumPy or SciPy) for data filtering and preprocessing, a machine learning framework (such as TensorFlow or PyTorch) for state analysis, and a generative information generation model (such as a large language model API) capable of transforming analytical results and prompt sentences into natural language. Optional software for multimodal emotion recognition (including image processing, voice analysis, and motion detection) may be incorporated to increase the accuracy of the state estimation.

The system operates as follows. The terminal collects raw biological signals from the subject using the biological signal acquisition device, digitizing and buffering the signal data. The terminal is configured to transmit the collected data securely to the server using an encryption-based communication method. The server receives and reconstructs the data, conducts preprocessing by filtering noise and artifacts, and formats the data for analysis. The server executes the machine learning model to infer the physical, cognitive, or emotional state of the subject.

In embodiments incorporating multimodal analysis, the server additionally receives sensor data such as facial images, voice recordings, and motion information to enhance the estimation of the subject's state. These data streams are integrated using conventional or custom fusion algorithms.

Once the state analysis is completed, the processor constructs a prompt sentence summarizing the analytical result and, if available, supplemental emotional information. This prompt is input to the generative information generation model, which outputs a natural language message comprehensible by the intended user (for example, a caregiver or store clerk). The natural language output is then securely transmitted to the terminal and presented clearly on the display device, allowing the user to understand and respond appropriately to the subject's state.

For example, in a retail store, a customer's emotional state can be determined by measuring brain wave data using a wearable EEG device. The analyzed result, such as “excited,” is input into a generative AI model with a prompt sentence like:

Emotion analysis result: Excited

Generate a natural language message expressing this state as it might be felt by a customer in a store.

The generative AI model may produce the following message:

The customer is excited; she seems to have found something special.

This message is transmitted to the clerk's smart glasses display, enabling the clerk to approach the customer with a personalized greeting.

In a healthcare setting, for example, the terminal may detect a state such as “anxious” and generate the following prompt sentence:

Emotion analysis result: Anxiety

Generate a natural language message expressing this feeling from the perspective of the user.

The generative model outputs:

I am anxious; I have something on my mind.

This natural language message is displayed on a caregiver's monitor or smart glasses, providing immediate and actionable insight into the patient's unspoken needs.

By employing widely available hardware and software technologies, including secure data transmission protocols, signal processing libraries, machine learning frameworks, and generative AI models, the invention can be efficiently implemented across various use cases where real-time understanding of a subject's biological or emotional state is desired.

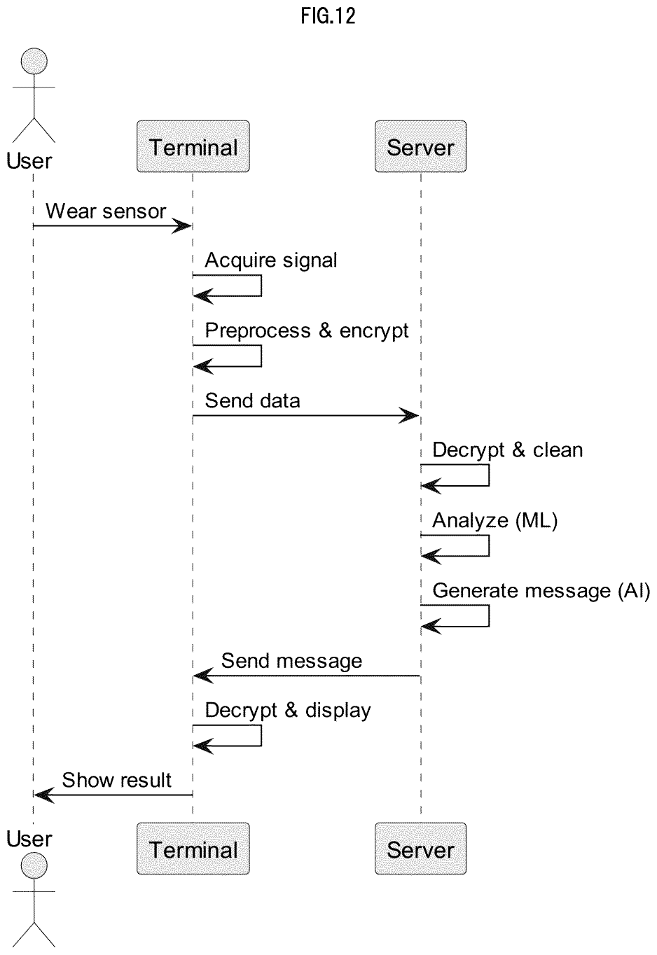

The following describes the processing flow using FIG. 12.

### Step 1:

The terminal acquires biological signal data from the user by activating the biological signal acquisition device, such as an EEG headset or sensor-equipped wearable. The input to this step is the user's raw physiological signals, captured as analog electrical activity. The terminal digitizes the signals using an analog-to-digital converter and buffers the resulting digital signal data. The output of this step is a sequence of digitized biological signal data stored in the terminal's memory.

### Step 2:

The terminal packages the buffered biological signal data into data packets suitable for transmission. The input for this step is the digitized biological signal data. The terminal applies encryption to each data packet using an encryption-based communication protocol, such as HTTPS, to ensure secure transmission. The output of this step is a set of encrypted data packets.

### Step 3:

The terminal transmits the encrypted data packets to the server via a secure network connection. The input is the set of encrypted data packets, and the output is the transmission of these packets to the server endpoint.

### Step 4:

The server receives the encrypted data packets and reconstructs the original biological signal data by decrypting and reassembling the packets. The input is the stream of encrypted data packets. The server performs data decryption, error checking, and packet ordering. The output of this step is the reconstructed, decrypted sequence of biological signal data ready for processing.

### Step 5:

The server preprocesses the reconstructed biological signal data by applying signal processing techniques, such as filtering (using SciPy or NumPy libraries) to remove noise and artifacts, and normalizing the data. The input to this step is the decrypted biological signal data. The server executes filtering algorithms, normalization routines, and performs artifact separation if necessary. The output is a cleaned, preprocessed biological signal dataset formatted for analysis.

### Step 6:

The server analyzes the preprocessed biological signal data by inputting it into a machine learning model, for example, a neural network built with TensorFlow or PyTorch. The input for this step is the cleaned biological signal data. The server executes model inference, identifying specific states such as emotional or cognitive conditions. The output is a set of analysis results, including predicted state labels and probability scores.

### Step 7:

The server optionally acquires and analyzes additional sensor data such as image information, voice recordings, or motion data to supplement the state estimation. The input to this step is multimodal sensor data collected from the terminal or related sensors. The server processes each data type with suitable algorithms (e.g., facial emotion recognition, voice analysis), fuses the results with the original analysis, and produces a refined state assessment. The output is an enhanced set of state analysis information.

### Step 8:

The server constructs a prompt sentence summarizing the derived analytical results and supplemental emotion information, then inputs this prompt into a generative information generation model. The input is the combined analysis results and a structured prompt sentence. The server queries the generative AI model, which processes the input and generates a natural language message expressing the state in human-understandable terms. The output is a natural language message text that explains the user's condition.

### Step 9:

The server encrypts and transmits the generated natural language message to the terminal via the secure communication protocol. The input is the natural language message, and the output is the encrypted message transmission to the terminal.

### Step 10:

The terminal receives, decrypts, and reconstructs the transmitted natural language message from the server. The input is the encrypted natural language message, and the output is the clear, readable message text stored for display.

### Step 11:

The terminal displays the received natural language message on its output device, such as smart glasses, a tablet, or a monitor. The input is the decrypted, generated message text. The terminal presents this information visually to the user, such as a caregiver or staff member. The output is the visible message on the device display, enabling the user to recognize and act upon the subject's emotional or cognitive state.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

In clinical and caregiving environments, it is often difficult to accurately understand the emotional and cognitive states of individuals, especially when they are unable to communicate verbally or express themselves clearly. Conventional technologies for biosignal data acquisition and analysis are commonly inadequate in providing secure, precise, and real-time interpretation of such states. Furthermore, there is a significant need for systems that can not only analyze complex biosignal data but also transform those analytical results into intuitive natural language messages. Additionally, the confidentiality and integrity of sensitive biosignal data during communication remains a challenge, along with the requirement for high-quality noise removal in preprocessing steps.

The specific processing by the specific processing unit 290 of the data processing device 12 in Example 2 is realized by the following means.

The present invention provides a server including a processor configured to collect biosignal data from a biosignal acquisition device, preprocess the collected biosignal data, analyze the preprocessed data using a machine learning algorithm to determine an emotional or cognitive state, enhance the analysis results through an emotion estimation engine, generate a prompt sentence based on the analysis and supplementary information, input this prompt to a generative artificial intelligence model to generate a natural language message, and display the message on an information display device. The processor is further configured to perform secure communication and advanced noise-removal including frequency component extraction during preprocessing. This enables secure, accurate, and real-time transformation of biosignal data into easily understandable natural language messages, allowing healthcare or caregiving staff to better understand and respond to the emotional and cognitive needs of individuals.

The term “biosignal data” refers to physiological electrical signals generated by a biological subject, such as brainwaves, that are measured by an electronic device for subsequent analysis.

The term “biosignal acquisition device” refers to an electronic apparatus or instrument configured to detect, measure, and collect biosignal data from a subject, such as an EEG headset or similar physiological sensor.

The term “processor” refers to a processing unit, such as a microprocessor or central processing unit, which executes instructions for data processing, analysis, and control within the system.

The term “preprocessing” refers to a set of data processing operations, including noise removal and data format conversion, that transform raw biosignal data into a format suitable for further analysis by computational models.

The term “noise removal” refers to the processing of acquired biosignal data to eliminate irrelevant or undesirable components, such as artifacts or interference, often by applying filtering algorithms.

The term “data format conversion” refers to the transformation of biosignal data from its raw initial state to a structured digital format suitable for computational analysis, such as conversion into digital arrays.

The term “machine learning algorithm refers to a computerized technique, such as a neural network, which processes input data and infers complex patterns or characteristics, including emotional or cognitive states.

The term “emotional or cognitive state” refers to the psychological condition or mental status of a subject, such as feelings, moods, or thoughts, as inferred from biosignal data.

The term “emotion estimation processing engine” refers to a computational module that receives and further analyzes results from a machine learning algorithm to enhance or supplement the determination of an emotional or cognitive state.

The term “prompt sentence” refers to a structured input statement composed for use with a generative artificial intelligence model, encapsulating system analysis results and relevant supplementary information.

The term “generative artificial intelligence model” refers to a data-driven model, trained on large datasets, that is capable of producing natural language output based on input data and prompts, such as a language generation neural network.

The term “natural language message” refers to a textual expression in human language, generated by computational processes, intended to communicate analytical results in a manner understandable to users.

The term “information display device” refers to any hardware, such as a monitor or screen, configured to visually present messages or data to users.

The term “encrypted communication protocol” refers to a data transmission standard or method that ensures security, confidentiality, and integrity of data through encryption during transfer between system components.

An embodiment of the invention will be described in detail below.

The system includes at least a terminal, a server, and one or more biosignal acquisition devices. The biosignal acquisition device, for example, an EEG headset or other physiological sensor, is attached to the user who may be a patient or subject requiring monitoring. This device is configured to detect and collect biosignal data, such as brainwave signals, which represent physiological electrical activity.

The terminal is implemented using computer hardware, such as a mobile device, tablet, or dedicated embedded system. The terminal communicates wirelessly with the biosignal acquisition device using a protocol such as Bluetooth. The terminal temporarily stores the received biosignal data in its internal memory or buffer for subsequent transmission.

The terminal securely transmits the collected biosignal data to the server using an encrypted communication protocol, for example, Transport Layer Security (TLS). The server is realized by a general-purpose computer system or a cloud server, including a processor, memory, network interfaces, and storage devices.

The server is equipped with software modules to preprocess received biosignal data. Software such as Python with libraries like NumPy and SciPy is used to perform noise removal and data format conversion, which may include the application of a band-pass filter to extract a desired frequency range (such as 4 Hz to 40 Hz). The preprocessed data is converted into a suitable digital array format for analysis.

The server then analyzes the preprocessed biosignal data using a machine learning algorithm. Examples include neural network models such as LSTM (Long Short-Term Memory) or CNN (Convolutional Neural Network), which may be implemented through machine learning frameworks such as PyTorch or TensorFlow. The machine learning model is trained in advance using a large dataset of biosignal data labeled with various emotional or cognitive states. The server infers the current emotional or cognitive state from the input data, for example, identifying the state as “anxiety” or “calm.”

The server further refines the analysis by inputting the results into an emotion estimation processing engine. This may be a distinct software module, such as an emotion estimation SDK or a custom module, that supplements and increases the precision of the detected emotion or cognitive state, providing composite or more nuanced information (such as “strong anxiety and slight confusion”).

Subsequently, the server generates a prompt sentence that incorporates the supplemented analytical results and any relevant additional information. For example, a prompt sentence may be:

“Given the following EEG data interpretation and emotion engine results, generate a natural language message representing the user's feelings and thoughts: Emotion: anxiety, Details: strong anxiety and slight confusion.”

The server then inputs this prompt sentence into a generative artificial intelligence model, such as a neural network based natural language generation model, which may be provided by a cloud-based API. The generative AI model produces a natural language message, such as:

“I am feeling very anxious and a little confused. Something doesn't feel right.”

The server transmits the generated message securely to the terminal using the encrypted protocol. The terminal receives the message, decodes it, and displays the natural language message on an information display device, such as a monitor or a screen. The caregiver or healthcare worker reads the message and is able to quickly understand the patient's emotional or cognitive state.

For example, if a non-communicative patient's biosignal data is collected and processed as described above, and the detected state is “strong anxiety with slight confusion,” the monitor on the terminal provides a message like “I am feeling very anxious and a little confused.” This enables the caregiver to promptly deliver suitable care or reassurance based on the patient's actual state.

An example of a prompt sentence used in this system is:

“Please analyze the following EEG signals and emotion engine results, and convert them into a natural language message expressing the user's current mental state: EEG Data: [filtered signals], Emotion: anxiety, Details: strong anxiety with slight confusion.”

This system may be constructed using widely available hardware such as general-purpose computers, standard mobile devices, and commercial biosignal acquisition equipment, in combination with software including encryption libraries, signal processing toolsets, and open-source or commercially available machine learning and generative artificial intelligence frameworks.

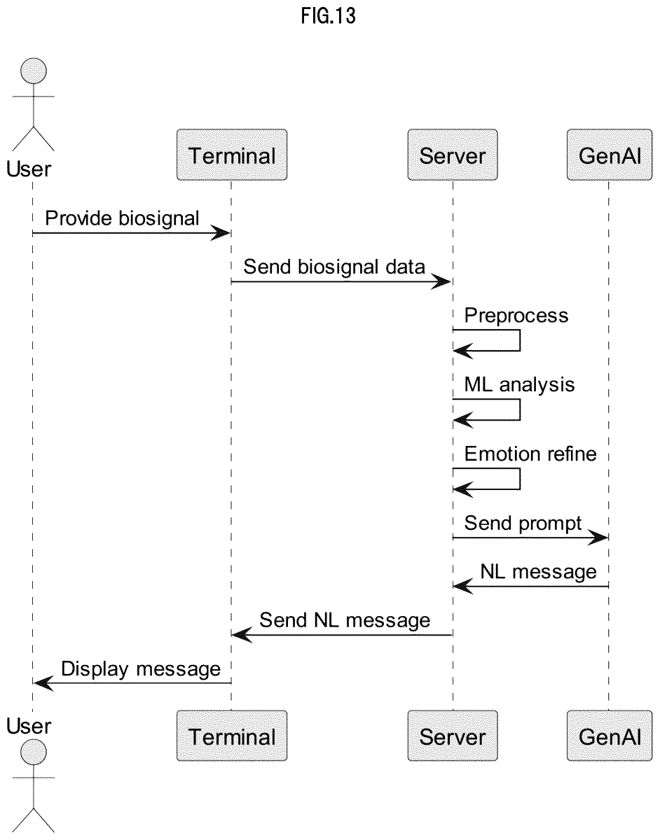

The following describes the processing flow using FIG. 13.

### Step 1:

The terminal obtains biosignal data from the user via a biosignal acquisition device, such as an EEG headset. The input is the raw electrical signals captured by the device in real time. The terminal processes the incoming signals by receiving them through a Bluetooth interface and temporarily storing the data in its local buffer. The output is a chunk of raw biosignal data ready for secure transmission.

### Step 2:

The terminal transmits the collected biosignal data to the server through an encrypted communication protocol, such as TLS. The input is the buffered raw biosignal data. The terminal performs data serialization, encrypts the data, and sends it to the server over a secure network connection. The output is the delivery of encrypted biosignal data packets to the server.

### Step 3:

The server receives and verifies the integrity of the biosignal data using the encrypted protocol. The input is the encrypted biosignal data packets. The server decrypts the packets, checks for errors, and reconstructs the raw biosignal data stream. The output is a verified, decrypted biosignal data set ready for preprocessing.

### Step 4:

The server preprocesses the biosignal data to enhance its quality for analysis. The input is the verified, raw biosignal data. The server applies signal processing operations, such as noise removal and frequency filtering (e.g., using a band-pass filter to extract the 4-40 Hz range), and converts the data into a standardized array format suitable for analysis. The output is a clean, normalized, and formatted biosignal data array.

### Step 5:

The server analyzes the preprocessed biosignal data using a machine learning algorithm, such as a neural network model (LSTM or CNN). The input is the preprocessed biosignal data array. The server loads the relevant trained model, feeds the data to the prediction engine, and executes inference to extract features and determine the probable emotional or cognitive state. The output is an initial label or set of values representing the detected state (e.g., “anxiety” or “calm”), possibly with a confidence score.

### Step 6:

The server enhances the analytical results with an emotion estimation processing engine for greater precision. The input is the analysis result from the machine learning model. The server passes the initial label and any extracted features to the emotion engine, which refines the result by combining more nuanced or composite information. The output is a detailed emotional or cognitive state descriptor with supplementary data.

### Step 7:

The server generates a prompt sentence for a generative AI model, incorporating the refined analysis and additional data. The input is the detailed emotional or cognitive state and supplementary information. The server formats this information as a structured natural language prompt. The output is a prompt sentence ready for submission to the generative AI model.

### Step 8:

The server submits the prompt sentence to the generative AI model and receives a natural language message. The input is the prompt sentence. The server transmits the prompt through the model's API, which processes the request and generates a user-friendly message expressing the emotional or cognitive state. The output is the generated natural language message.

### Step 9:

The server transmits the generated natural language message back to the terminal using an encrypted communication protocol. The input is the natural language message. The server serializes, encrypts, and sends the message over the secure channel. The output is a decrypted natural language message received by the terminal.

### Step 10:

The terminal displays the received natural language message on an information display device, such as a monitor. The input is the natural language message received from the server. The terminal decodes and renders the text on-screen, possibly triggering an audio or visual alert for the caregiver. The output is the visible, easy-to-understand message presented to the caregiver or healthcare worker, enabling an appropriate response to the user's needs.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

In industrial and other operational environments where humans interact with automated machinery, it is difficult to accurately and promptly communicate the emotional and cognitive states of users to machines, especially in real time. Existing systems often lack the capability to interpret biosignal data, such as brainwave signals, and convert them into actionable and understandable information for machinery or for feedback to users. Additionally, there is a need for secure and precise data handling, as well as effective bidirectional communication between users and automated systems to improve efficiency, safety, and user well-being.

The specific processing by the specific processing unit 290 of the data processing device 12 in Application Example 2 is realized by the following means.

The present invention provides a server including a processor configured to collect biosignal data acquired by a biosignal acquisition device, preprocess the collected biosignal data and convert it into a format suitable for analysis, analyze the preprocessed biosignal data using a machine learning model to determine a user state, enhance the precision of the state data, employ a generative artificial intelligence model to convert the state data or machinery feedback into natural language information, notify automated machinery of the converted information, collect processing status or abnormality information from the machinery, and output the converted natural language information to a display device using a secure communication protocol and noise removal processing during preprocessing. This enables accurate, real-time, and secure exchange of meaningful information between users and automated machinery, thereby improving efficiency, safety, and overall system performance.

The term “biosignal data” refers to physiological data acquired from a user, including but not limited to brainwave signals, that reflect the user's bodily or mental state.

The term “biosignal acquisition device” refers to a hardware apparatus or sensor configured to detect and measure biosignal data from a user.

The term “preprocessing” refers to a set of operations performed on raw biosignal data to remove noise, artifacts, or irrelevant information, and to convert the data into a format suitable for computational analysis.

The term “format suitable for analysis” refers to a data structure or representation that allows efficient and accurate processing by subsequent analytical algorithms or machine learning models.

The term “machine learning model” refers to a computational algorithm or a system that is trained using data to recognize patterns, classify information, or predict the user's physiological or emotional state based on input data.

The term “state of a user” refers to a condition or status of the user determined from biosignal data, including but not limited to emotional, cognitive, or physical states, such as stress, fatigue, or alertness.

The term “enhance the precision” refers to improving the accuracy, reliability, or resolution of the analyzed state data to enable more effective interpretation and response. The term “generative artificial intelligence model” refers to an artificial intelligence system capable of generating human-like natural language output or other forms of meaningful content based on received input data.

The term “input sentence” refers to structured textual information provided to the generative artificial intelligence model as a prompt for generating natural language content or messages.

The term “natural language information” refers to information expressed in human language, which is understandable to human users, generated by the generative artificial intelligence model.

The term “industrial automated machinery” refers to automated hardware devices used in industrial or operational settings that perform tasks or processes without direct human intervention.

The term “processing status or abnormality information” refers to operational data or messages from industrial automated machinery indicating current performance, state, error conditions, or system abnormalities.

The term “display device” refers to any hardware or interface capable of visually presenting information, including natural language information, to a user.

The term “communication protocol compliant with information security standards” refers to a data transmission method or system that meets defined criteria for confidentiality, integrity, and security of information.

The term “noise removal processing” refers to computational techniques or algorithms applied to biosignal data to eliminate or reduce unwanted signals, artifacts, or interference.

One embodiment for implementing the invention described in the claims is as follows.

A user wears a biosignal acquisition device, such as a brainwave sensor, on their head to allow continuous acquisition of physiological signals while working in an industrial environment. These signals, such as brainwave data, are transmitted in real time from the biosignal acquisition device to a terminal device. The terminal may be a mobile computing device or an embedded hardware unit, including temporary local storage and communication capability. The terminal reliably collects raw biosignal data and then transmits the data using a communication protocol compliant with information security standards, for example, TLS (Transport Layer Security), to a server for further processing.

The server includes a processor configured to preprocess the received biosignal data. In one configuration, the server applies noise removal processing using software libraries such as SciPy or MATLAB to filter out environmental or physiological artifacts (such as eye blinks and electrical interference). The preprocessed data are converted into a format suitable for analysis, such as normalized arrays.

Once the biosignal data have been cleaned and preprocessed, the server analyzes the data using a machine learning model. This model, which may be implemented in a software package such as TensorFlow or PyTorch, processes the biosignal data to determine the user's state, including emotional or cognitive conditions such as stress, fatigue, or focus. In some embodiments, the server further enhances the precision of this state determination using an analysis algorithm or engine specializing in emotional state evaluation.

The analyzed and refined state data are then provided as an input sentence (prompt sentence) to a generative artificial intelligence model hosted on the server. This generative AI model, which may be based on open large language model architectures (such as GPT models), converts the input analysis into natural language information. For example, the generative AI model could generate natural language sentences such as “I am feeling stressed and need a break,” or “Fatigue level is high, please slow down the process.”

The server notifies the industrial automated machinery of the generated natural language information via an interface. The machinery may then execute predefined actions, such as adjusting operation speed, activating safety protocols, or sending an acknowledgment.

The server also collects processing status or abnormality information from the industrial automated machinery in real time. This information, often technical or coded, is again converted into human-understandable natural language using the generative AI model. For instance, the technical status “Cooling system abnormality detected” is processed into the natural language “The robot detected a problem in the cooling system. Please carry out maintenance.”

The resulting natural language information is sent by the server to a display device, such as a head-mounted display worn by the user or a screen installed in the working environment. This display enables the user to receive timely and easily comprehensible feedback from the machinery.

The hardware employed in this embodiment may include, but is not limited to, a biosignal acquisition device (for example, a brainwave sensor), a terminal device (such as a mobile computer), a head-mounted display, and an interface device for industrial automated machinery. The software may include signal processing toolkits (such as SciPy or MATLAB), machine learning frameworks (such as TensorFlow or PyTorch), an emotion analysis engine, and a generative AI model.

A typical prompt sentence provided to the generative AI model might be:
“The user's stress level is high and fatigue level is moderate. Generate a notification message for a factory robot.”

Another prompt example for converting machine feedback to the user might be:
“Convert this robot status into easily understood English for a factory worker: Cooling system abnormality detected.”

Through these components and processing steps, the invention enables real-time, secure, and accurate bidirectional communication between users and automated machinery using biosignal data, artificial intelligence, and natural language generation. This configuration improves operational safety, workplace efficiency, and the well-being of users in environments with industrial automated machinery.

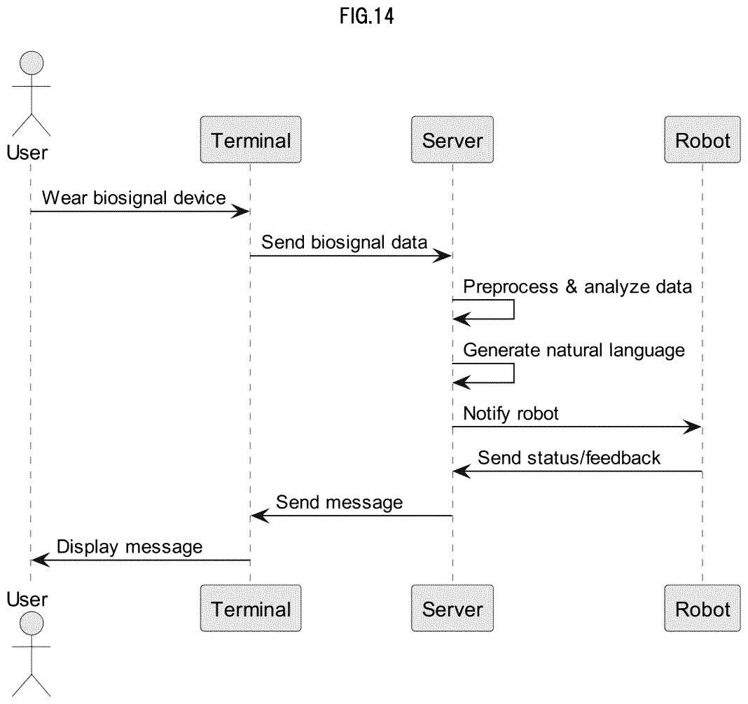

The following describes the processing flow using FIG. 14.

### Step 1:

User wears a biosignal acquisition device, such as a brainwave sensor, on their head prior to starting work. The device establishes contact with the scalp to ensure accurate capture of physiological signals.

Input: No data input (physical setup)

Output: User is ready for biosignal data acquisition.

### Step 2:

Terminal continuously receives biosignal data (such as brainwave signals) from the biosignal acquisition device in real time and temporarily stores the raw data in its local buffer. Input: Analog biosignal data from the biosignal acquisition device

Data processing: Terminal digitizes analog signals and stores them in an internal buffer.

Output: Buffered digital biosignal data in the terminal memory.

### Step 3:

Terminal transmits the buffered biosignal data to the server using a secure communication protocol (such as TLS) to ensure data confidentiality and integrity during transfer.

Input: Buffered digital biosignal data in the terminal memory

Data processing: Terminal encrypts the buffer and packages biosignal data into transmission packets.

Output: Encrypted packets containing biosignal data sent to the server.

### Step 4:

Server receives the encrypted biosignal data from the terminal, decrypts it, and preprocesses the data by applying noise removal processing (using, for example, a band-pass filter implemented with SciPy) to eliminate artifacts. The server then normalizes and segments the data for further analysis.

Input: Encrypted biosignal data packets

Data processing: Server performs decryption, applies noise removal algorithms, and normalizes the data array.

Output: Cleaned and normalized biosignal data prepared for analysis.

### Step 5:

Server analyzes the cleaned and normalized biosignal data using a machine learning model implemented with a platform such as TensorFlow or PyTorch. The server interprets the data to determine the user's state (e.g., stress, fatigue, focus) and then uses an emotion analysis engine to enhance the precision of the result.

Input: Cleaned and normalized biosignal data

Data processing: Server computes machine learning predictions and refines results with an emotion analysis algorithm.

Output: Enhanced state data (example: stress=0.87, fatigue=0.65, focus=0.42).

### Step 6:

Server generates a prompt sentence based on the enhanced state data and inputs it into a generative AI model (such as a large language model). The model converts quantitative user state data into human-friendly natural language information.

Input: Enhanced state data

Data processing: Server forms an input sentence for the generative AI model (for example: “The user's stress level is high and fatigue level is moderate. Generate a message for the machinery.”) and requests the generative AI model to generate corresponding text.

Output: Natural language information generated by the generative AI model (for example: “I am feeling stressed and need a break.”).

### Step 7:

Server sends the generated natural language information to the industrial automated machinery via an interface. The machinery interprets the notification and executes actions according to predefined protocols.

Input: Natural language message for machinery

Data processing: Server formats and transmits the text to the industrial automated machinery using the interface.

Output: Industrial automated machinery receives and processes the natural language message.

### Step 8:

Server collects feedback or status information from the industrial automated machinery, such as status codes or abnormality alerts, through the interface. The server then prepares this information for user-friendly feedback.

Input: Machine status or abnormality information

Data processing: Server collects, logs, and formats raw messages from the machinery.

Output: Raw status or abnormality information ready for language conversion.

### Step 9:

Server inputs the collected feedback or status information into the generative AI model, using a suitable prompt sentence, to transform it into a human-understandable natural language message for the user.

Input: Raw machine status information

Data processing: Server creates a prompt (for example: “Convert this status into English for the worker: Cooling system abnormality detected.”) and uses the generative AI model to generate plain language feedback.

Output: Natural language information conveying machinery status to the user.

### Step 10:

Terminal receives the natural language message from the server and displays it on the display device, such as a head-mounted display worn by the user, so the user can recognize and react to important system messages.

Input: Natural language information for the user

Data processing: Terminal renders the message on the display device in a clear and easily visible format.

Output: User visually receives feedback or warnings in natural language through the display device.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

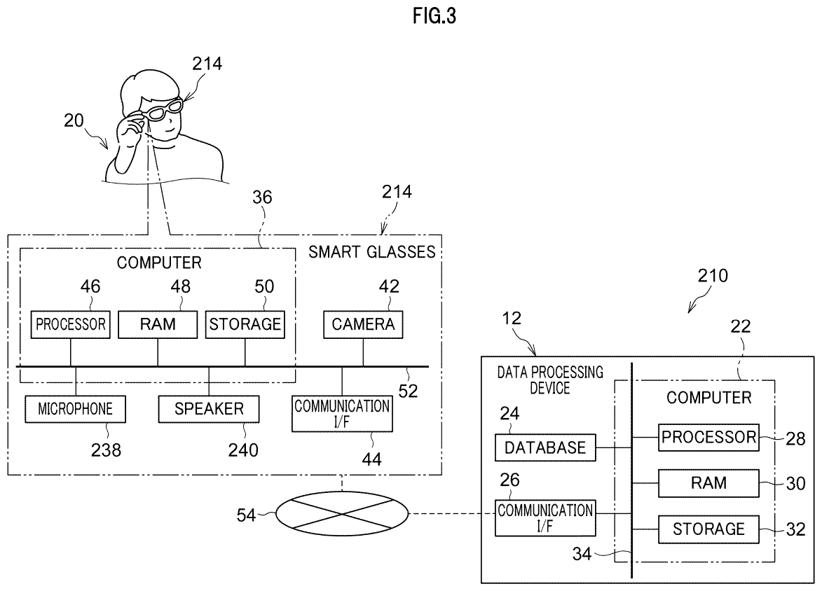

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

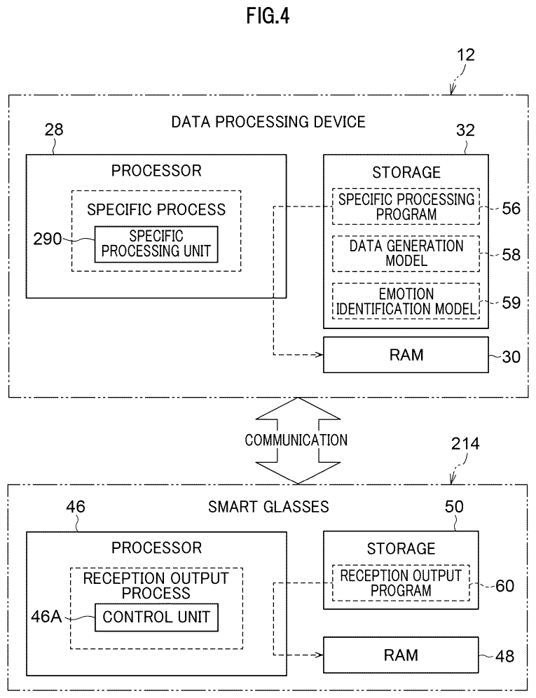

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

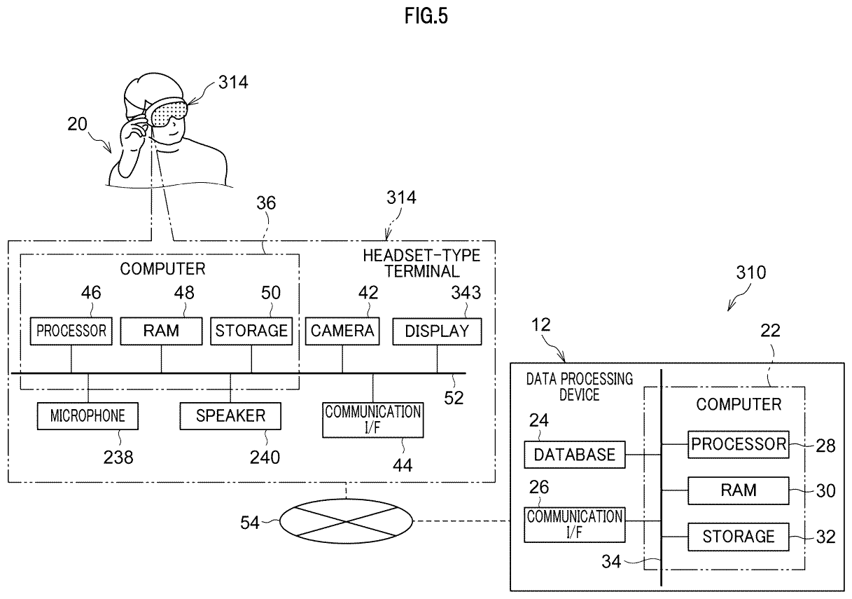

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

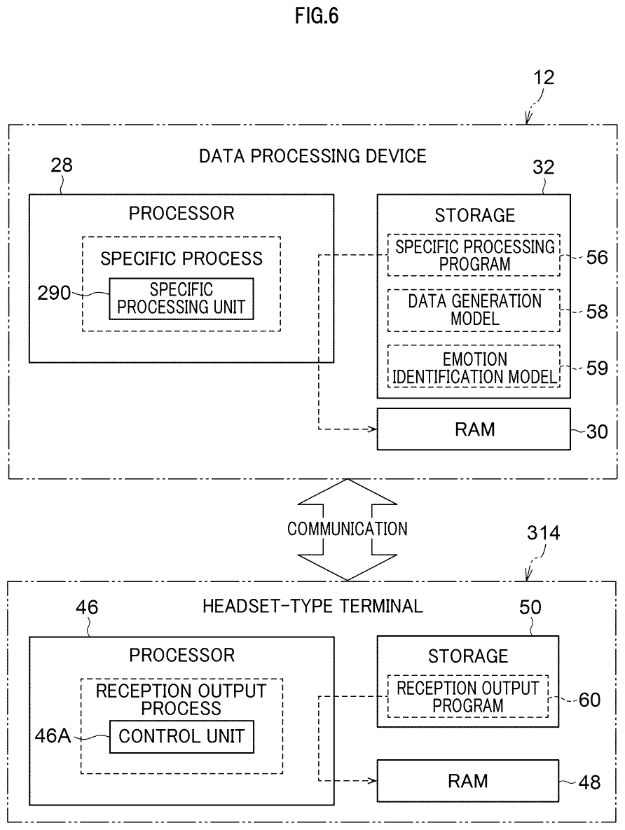

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

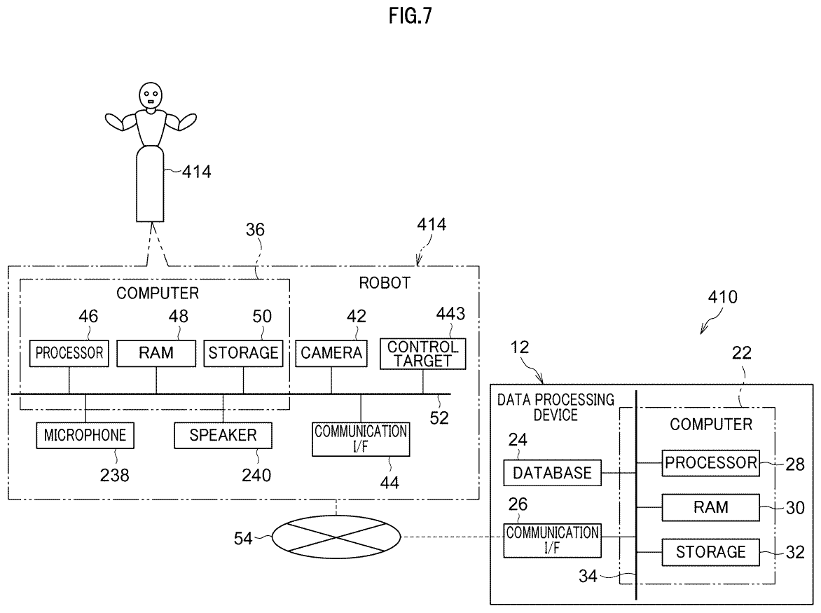

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

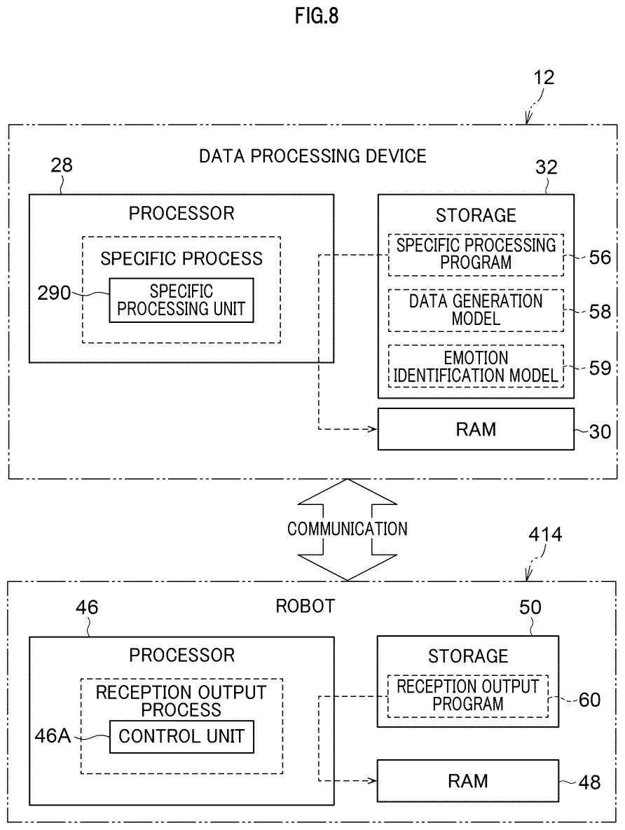

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

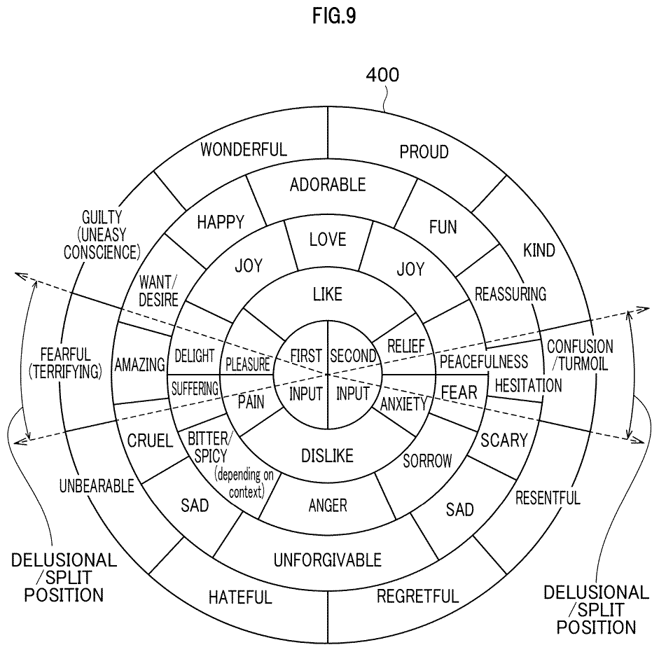

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

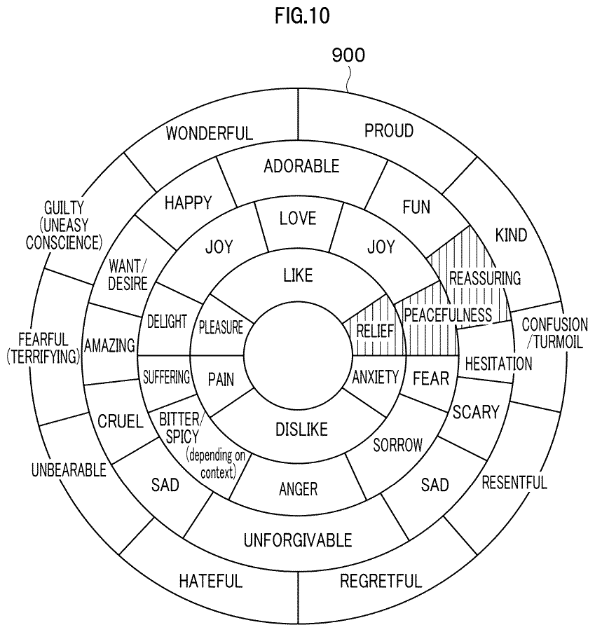

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### (Supplementary 1)

A system including a processor,
wherein the processor is configured to:
collect biological signal data obtained by a biosignal acquisition device,
temporarily store the collected biological signal data in a storage device and manage it as time-series data,
encrypt the collected biological signal data and transmit it to an information processing apparatus via a secure communication protocol,
verify the integrity and decrypt the biological signal data received by the information processing apparatus,
perform a preprocessing including noise removal on the biological signal data and convert the data into an analyzable data format,
input the preprocessed biological signal data into a machine learning model to classify the mental state or cognitive state of a user,
input a prompt sentence including the classified mental state or cognitive state into a generative artificial intelligence model to convert the result into natural language, and
display the generated natural language message on a display device.

### (Supplementary 2)

The system according to supplementary 1,
wherein the processor is configured to generate a prompt sentence corresponding to the classified mental state or cognitive state as the input for the generative artificial intelligence model.

### (Supplementary 3)

The system according to supplementary 1,
wherein the processor is configured to manage the recording, transmission, retransmission, and reacquisition of biological signal data during temporary storage and preprocessing, including handling retransmission in the event of communication failures.

### Application Example 1

### (Supplementary 1)

A system including a processor,
wherein the processor is configured to:
collect biological signal data obtained by a biological signal acquisition device,
preprocess the collected biological signal data to convert it into a data format suitable for analysis,
apply signal processing techniques to the preprocessed biological signal data for data cleanup,
input the preprocessed and cleaned biological signal data into a machine learning model to analyze a state of a subject,
additionally acquire and analyze multiple types of sensor data including image information, voice information, and body motion information to enhance accuracy of the state analysis,
input the analyzed state information and supplementary emotional information as a prompt sentence into a generative information generation model to convert the information into natural language, and
display the converted natural language information on an output device.

### (Supplementary 2)

The system according to supplementary 1,
wherein the processor is configured to transmit and receive at least one of the collected biological signal data and the generated natural language information using an encryption-based communication method.

### (Supplementary 3)

The system according to supplementary 1,
wherein the processor is configured to remove noise components and disturbances from the biological signal data by using a signal processing algorithm in the preprocessing stage.

### Example 2

### (Supplementary 1)

A system including a processor,
wherein the processor is configured to:
collect biosignal data acquired by a biosignal acquisition device,
perform preprocessing on the collected biosignal data, including noise removal and data format conversion, to convert the biosignal data into a format suitable for analysis,
input the preprocessed biosignal data into a machine learning algorithm to analyze an emotional or cognitive state,
enhance and supplement the analyzed emotional or cognitive state by using an emotion estimation processing engine,
generate a prompt sentence including the supplemented analysis result and additional information, input the prompt sentence into a generative artificial intelligence model to generate a natural language message, and
display the generated natural language message on an information display device.

### (Supplementary 2)

The system according to supplementary 1,
wherein the processor is configured to transmit or receive the collected biosignal data and the generated natural language message using an encrypted communication protocol.

### (Supplementary 3)

The system according to supplementary 1,
wherein the processor is configured to perform noise removal processing including frequency component extraction during the preprocessing stage.

### Application Example 2

### (Supplementary 1)

A system including a processor,
wherein the processor is configured to:
collect biosignal data acquired by a biosignal acquisition device;
preprocess the collected biosignal data and convert the data into a format suitable for analysis;
analyze the preprocessed biosignal data by inputting the data into a machine learning model to determine a state of a user;
enhance the precision of the state data based on the analysis result;
input the enhanced state data as an input sentence to a generative artificial intelligence model and convert the state data into natural language information;
notify industrial automated machinery of the converted natural language information; collect processing status or abnormality information from the industrial automated machinery;
input the collected processing status or abnormality information as an input sentence to the generative artificial intelligence model and convert the information into natural language information; and
output the converted natural language information to a display device.

### (Supplementary 2)

The system according to supplementary 1,
wherein the processor is configured to transmit the collected biosignal data using a communication protocol compliant with information security standards.

### (Supplementary 3)

The system according to supplementary 1,
wherein the processor is configured to apply noise removal processing during the preprocessing phase.

What is claimed is:

1. A system comprising a processor,
wherein the processor is configured to:
collect EEG data obtained by an EEG measurement device;
preprocess the collected EEG data and convert the data into a format suitable for analysis;
input the preprocessed EEG data into a deep learning model to analyze emotions or thoughts;
convert the analysis result into natural language using a generative AI model; and display the converted natural language.

2. The system of claim 1, wherein the processor is further configured to transmit the collected EEG data using a secure communication protocol.

3. The system of claim 1, wherein the processor is further configured to apply a noise removal filter during the preprocessing stage.
